(19) United States
(12) Patent Application Publication
(10) Pub. No.: US 2026/0369378 A1
(43) Pub. Date: Nov. 19, 2026

(54) FRACTIONATION SYSTEMS AND METHODS FOR RADIOACTIVE MICROSPHERE DOSES FOR CANCER THERAPY

(71) Applicant: Boston Scientific Scimed, Inc., Maple Grove, MN (US)

(72) Inventors: Kyle True, Minneapolis, MN (US); Katrina Marie Daley, Corcoran, MN (US); Zachary Nickle, Minneapolis, MN (US); Rebecca Jean Busacker, Chanhassen, MN (US); Michael Fandel, Plymouth, MN (US); Ramon Libarnes, Dayton, MN (US); McKenzie Dudley, St. Louis Park, MN (US); Scott McGhee, Ottawa (CA)

(21) Appl. No.: 19/676,433

(22) Filed: May 13, 2026

Related U.S. Application Data
(60) Provisional application No. 63/807,287, filed on May 16, 2025.

(51) Int. Cl. G16H 20/17; A61K 51/12

(52) U.S. Cl. CPC G16H 20/17; A61K 51/1255

(57) ABSTRACT

Embodiments herein relate to fractionation systems for preparing doses of radioactive microspheres and related methods. For example, in an embodiment an automated dose fractionation system for radioactive microspheres for cancer treatment is included having a control circuit and a carrier fluid pump configured to be controlled by the control circuit. The system also includes a master vial receiver configured to engage with a master vial containing the radioactive microspheres. The system also includes a mixing container disposed over the master vial receiver. The automated dose fractionation system can be configured to execute a series of pre-programmed carrier fluid flush cycles to transfer microspheres from the master vial into the mixing container. Other embodiments are also included herein.

This application claims the benefit of U.S. Provisional Application No. 63/807,287, filed May 16, 2025, the content of which is herein incorporated by reference in its entirety.

### FIELD

Embodiments herein relate to fractionation systems for preparing doses of radioactive microspheres for cancer therapy and related methods.

### BACKGROUND

Radioactive microspheres have been developed as a means of treating cancer by delivering targeted radiation therapy directly to tumors. These microspheres, which contain therapeutic radioisotopes, can be administered using systems that infuse them through a delivery catheter for precise delivery to the target treatment site. For example, Yttrium-90 (Y-90) glass microspheres can be delivered into or adjacent to a tumor through a microcatheter placed into an artery that supplies blood to the tumor. The beta radiation emitted by the Y-90 can exert a local radiotherapeutic effect on the tumor. Other radioisotopes can also be used in some types of brachytherapy. This targeted therapeutic approach can maximize the radiation dose at the tumor site while minimizing exposure to healthy surrounding tissue. Despite ongoing advancements, challenges remain in improving the precision and adaptability of these systems across diverse clinical applications.

### SUMMARY

Embodiments herein relate to fractionation systems for preparing doses of radioactive microspheres for cancer therapy and related methods. In a first aspect, an automated dose fractionation system for radioactive microspheres for cancer treatment can be included having a control circuit, a carrier fluid pump controlled by the control circuit, a master vial receiver configured to engage with a master vial containing the radioactive microspheres, and a mixing container. The mixing container can be disposed over the master vial receiver. The automated dose fractionation system can be configured to execute a series of pre-programmed carrier fluid flush cycles to transfer microspheres from the master vial into the mixing container.

In a second aspect, in addition to one or more of the preceding or following aspects, or in the alternative to some aspects, the system can further include an agitation system, wherein at least a portion of the agitation system can be disposed within the mixing container.

In a third aspect, in addition to one or more of the preceding or following aspects, or in the alternative to some aspects, the agitation system can be configured to rotate and periodically change rotation direction.

In a fourth aspect, in addition to one or more of the preceding or following aspects, or in the alternative to some aspects, the carrier fluid flush cycles include injecting the carrier fluid into the master vial using the carrier fluid pump and then withdrawing a suspension of carrier fluid and radioactive microspheres out of the master vial and into the mixing container.

In a fifth aspect, in addition to one or more of the preceding or following aspects, or in the alternative to some aspects, the radioactive microspheres can include Y-90 glass microspheres.

In a sixth aspect, in addition to one or more of the preceding or following aspects, or in the alternative to some aspects, the radioactive microspheres have a density greater than three times the density of water.

In a seventh aspect, in addition to one or more of the preceding or following aspects, or in the alternative to some aspects, the mixing container can be disposed directly over the master vial receiver.

In an eighth aspect, in addition to one or more of the preceding or following aspects, or in the alternative to some aspects, can further include a fluid injection line, wherein the fluid injection line can be configured to extend into the master vial as held by the master vial receiver and be in fluid communication with the carrier fluid pump.

In a ninth aspect, in addition to one or more of the preceding or following aspects, or in the alternative to some aspects, can further include a suspension withdrawal line, wherein the suspension withdrawal line can be configured to extend into the master vial as held by the master vial receiver and be in fluid communication with the mixing container.

In a tenth aspect, in addition to one or more of the preceding or following aspects, or in the alternative to some aspects, can further include a holding container, wherein the holding container can be in fluid communication with the mixing container, and wherein the automated dose fractionation system can be configured to transfer a predetermined dose of microspheres from the mixing container to the holding container based on controlling a total volume of a suspension of microspheres and carrier fluid transferred from the mixing container to the holding container.

In an eleventh aspect, in addition to one or more of the preceding or following aspects, or in the alternative to some aspects, can further include a radiation sensor, wherein the radiation sensor can be configured to detect radiation within the holding container.

In a twelfth aspect, in addition to one or more of the preceding or following aspects, or in the alternative to some aspects, the automated dose fractionation system can further include a first radiation sensor, wherein the first radiation sensor can be configured to detect radiation within the mixing container, and a second radiation sensor, wherein the second radiation sensor can be configured to detect radiation in a holding container.

In a thirteenth aspect, in addition to one or more of the preceding or following aspects, or in the alternative to some aspects, can further include an output line, wherein the output line can be in fluid communication with a holding container and can be configured to convey the microspheres and carrier fluid from the holding container to a delivery catheter that connects to the automated dose fractionation system.

In a fourteenth aspect, in addition to one or more of the preceding or following aspects, or in the alternative to some aspects, the automated dose fractionation system can further include a delivery catheter connector, wherein the delivery catheter connector can be configured to provide a fluid communication link between the output line and a delivery catheter, and a connector hub bypass element, wherein the connector hub bypass element can be configured to reduce or eliminate dead space within and between the delivery catheter connector and the delivery catheter.

In a fifteenth aspect, a method of automated dose fractionation of radioactive microspheres for cancer treatment can be included. The method can include executing a series of pre-programmed carrier fluid flush cycles to transfer radioactive microspheres from a master vial into a mixing container that can be disposed over the master vial, agitating the radioactive microspheres and carrier fluid within the mixing container with an agitation system to maintain a suspension of radioactive microspheres and carrier fluid, and transferring a predetermined dose of microspheres from the mixing container to a holding container in one or more transfer steps based on controlling a total volume of a suspension of microspheres and carrier fluid transferred from the mixing container to the holding container.

In a sixteenth aspect, in addition to one or more of the preceding or following aspects, or in the alternative to some aspects, the method can further include rotating and periodically changing rotation direction of a component of the agitation system.

In a seventeenth aspect, in addition to one or more of the preceding or following aspects, or in the alternative to some aspects, the method can further include conveying the dose of microspheres and carrier fluid from the holding container to a delivery catheter.

In an eighteenth aspect, in addition to one or more of the preceding or following aspects, or in the alternative to some aspects, the method can further include transferring a second predetermined dose of microspheres from the mixing container to the holding container based on controlling a total volume of a suspension of microspheres and carrier fluid transferred from the mixing container to the holding container.

In a nineteenth aspect, in addition to one or more of the preceding or following aspects, or in the alternative to some aspects, the method can further include conveying the second dose of microspheres and carrier fluid from the holding container to a delivery catheter.

In a twentieth aspect, in addition to one or more of the preceding or following aspects, or in the alternative to some aspects, the method can further include detecting radiation within the holding container and/or detecting radiation within the mixing container.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures(FIGS.), in which:

FIG. 1 is a schematic view of a dose fractionation system in accordance with various embodiments herein along with a delivery catheter and a patient.

FIG. 2 is a schematic view of a dose fractionation system in accordance with various embodiments herein.

FIG. 3 is a diagram of operations in accordance with various embodiments herein.

FIG. 4 is a flowchart of operations in accordance with various embodiments herein.

FIG. 5 is a perspective view of components associated with the connection of a fractionation system herein with a delivery catheter in accordance with various embodiments herein.

FIG. 6 is a cross-sectional view of a master vial in accordance with various embodiments herein.

FIG. 7 is a schematic view of a master vial and a mixing container in accordance with various embodiments herein.

FIG. 8 is a schematic view of a mixing container in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, radioactive microspheres have been developed as a means of treating cancer by delivering targeted radiation therapy directly to tumors. Radioactive microspheres may be manufactured, placed in a master vial, and then shipped to a treatment site for clinical use.

In some cases, a clinician may desire to deliver the entire dose of radioactive microspheres in the master vial to a single treatment site during a single infusion event. However, in other cases, a clinician may desire to deliver a fraction of the entire dose of radioactive microspheres and then take an action such as, for example, inserting a new delivery catheter and then delivering another fraction or the remainder of the dose within the master vial. Thus, in some cases, there is a need to fractionate the total dose of radioactive microspheres from the master vial into multiple smaller doses. It will be appreciated, however, that there are various challenges associated with the accurate and efficient fractionation of doses.

In accordance with embodiments herein, automated dose fractionation systems for radioactive microspheres can be utilized in the context of cancer therapies to deliver precise fractionated doses of microspheres containing therapeutic radioisotopes directly to targeted treatment sites. The system can execute a series of pre-programmed carrier fluid flush cycles to transfer all radioactive microspheres from the master vial to the mixing container. In specific, the automated dose fractionation system herein can utilize a control unit that manages the execution of carrier fluid flush cycles. After transfer to the mixing container, the control unit can also manage operation of an agitation system to effectively maintain the suspension of the microspheres and carrier fluid in a homogeneous state in the mixing container. As such, the architecture of the system ensures control over the suspension homogeneity, mitigating the risk of uneven or inaccurate radiation dosage.

Systems herein can incorporate mechanisms for measurement and control to ensure the accurate fractionation of doses passing into a separate holding container while maintaining a stable suspension within the mixing chamber. In some embodiments herein, the transfer of precise amounts of radioactive microspheres from the mixing container to the holding container can be controlled using a volumetric control technique. In specific, the entire dose (e.g., a known quantity) of radioactive microspheres can first be transferred from the master vial to a mixing container. A known amount of carrier fluid can be combined with the radioactive microspheres in a well-mixed and homogenous suspension. At that point, each unit volume of suspension contains a known amount of radioactive microspheres. This allows a precise dose (or fraction) of microspheres to be transferred from the mixing container to a separate holding container (representing a fractionated dose for use with a single infusion event) by precisely controlling the volume that is transferred (in one transfer step or multiple transfer steps to achieve the desired fractionated dose) from the mixing container to the holding container. Embodiments herein can use this volumetric control technique in an automated manner.

As the suspension of microspheres and carrier fluid is withdrawn from the mixing chamber, if further carrier fluid is not added to the mixing chamber then the concentration within the mixing chamber stays constant and therefore the volume of suspension needed to be moved into the holding container to transfer a desired amount of microspheres also stays constant. However, if further carrier fluid is added to the mixing chamber as suspension is passed to the holding container, then the concentration of microspheres in the carrier fluid in the mixing chamber declines according to a dilution curve (like a decay curve) that can be exponential. Since volumes of carrier fluid added to the mixing chamber are known by the system, the dilution curve itself is known. In this scenario, the volume of suspension needed to be moved into the fractionation system to transfer a desired amount of microspheres increases but is a known value based on the dilution curve. As such, the system herein can calculate the volume of suspension that needs to be transferred to transfer a desired amount of microspheres into the fractionation system using the dilution curve.

From the holding container, the measured or fractionated dose can then be infused into the patient through a delivery catheter and then another dose can be queued up by the system for delivery by transferring an additional volume of suspension from the mixing container to the holding container.

Embodiments herein provide benefits in terms of addressing the challenges associated with dose customization, allowing for patient-specific treatment regimens tailored to the individual's therapeutic needs. Furthermore, the integration of a holding container within the automated dose fractionation system enables significant operational and safety advantages for the preparation and delivery of fractionated radioactive microsphere doses. Specifically, including the holding container as an intermediate reservoir located downstream of the mixing container permits decoupling of the fractionation processes from the actual dose administration event. This decoupling allows the system to complete the transfer and verification of a selected fractionated dose (by drawing a precisely measured volume of the homogeneously mixed suspension from the mixing container to the holding container) while permitting the operator or attending clinician to perform other procedural tasks or prepare the patient for treatment without requiring immediate administration of the dose. The holding container thereby acts as a functional holding point within the process flow, ensuring that the preparation of each fractionated dose is operationally independent from the timing of its administration.

Further, the holding container provides an isolated location within the system where the entire measured or fractionated dose can be temporarily maintained or “dwell” prior to initiation of infusion providing an opportunity for real-time validation of the measured radioactivity associated with the dose contained within the holding container using integrated system radiation sensor(s). In embodiments including one or more radiation sensors operatively associated with the holding container, the system can obtain a quantitative measurement of radioactivity that directly corresponds to the fractionated dose transferred. The measured activity within the holding container can then be compared against the prescribed dose or the value input by the clinician into the system via operator interface. If the measured activity and the prescribed or target dose correspond within acceptable operational tolerances, the system can provide a confirmation alert to the operator, thereby supporting positive confirmation prior to patient administration. Conversely, if a mismatch is detected, the system can alert the operator and inhibit further dose delivery until the discrepancy is resolved. By enabling independent and automated confirmation of each fractionated dose's radioactivity immediately prior to administration, the inclusion of the holding container and associated radiation sensors provides a robust safeguard mechanism to ensure dosing accuracy.

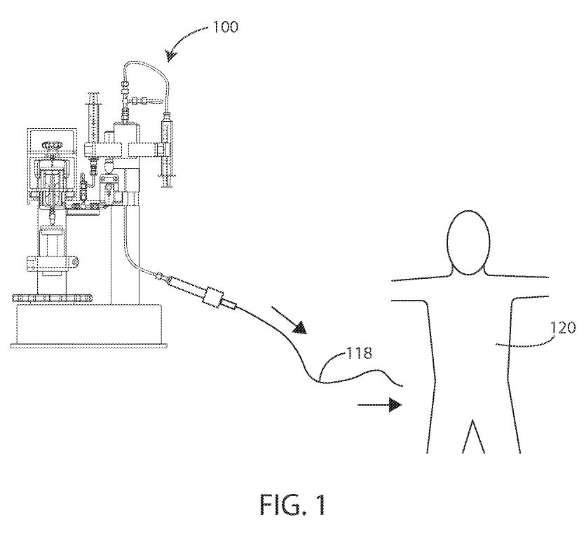

Referring now to FIG. 1, a schematic diagram illustrates a dose fractionation system configured for the treatment of cancer using radioactive microspheres. The system 100 is shown connected to a delivery catheter 118 that is configured to provide radioactive microspheres to a patient 120. This figure exemplifies an embodiment of the automated delivery configuration, emphasizing the integration of the dose fractionation system into clinical applications for targeted administration of therapeutic radioactive particles.

In various embodiments, the automated dose fractionation system 100 for radioactive microspheres used in cancer treatment is configured to perform a set of programmed carrier fluid flush cycles. These flush cycles facilitate the transfer of microspheres from a master vial to a mixing container. Specifically, the system can execute flush cycles by introducing a carrier fluid into the master vial via a carrier fluid pump, forming a suspension with the radioactive microspheres. This suspension is then extracted from the master vial, such as by using a pump, and transferred to the mixing container. Additionally, the automated system can transfer a specified dose of microspheres from the mixing container to a holding container. This transfer process is regulated by controlling the volume of the microsphere and carrier fluid suspension moved from the mixing container to the holding container.

In various embodiments, an automated dose fractionation system for radioactive microspheres used in cancer treatment, identified as system 100, includes the following components: a control circuit, a carrier fluid pump, a master vial receiver, and a mixing container. The carrier fluid pump is operated by the control circuit and facilitates the transfer of radioactive microspheres from a master vial into a mixing container by means of a series of pre-programmed carrier fluid flush cycles. The master vial receiver secures the master vial containing the microspheres, and the mixing container is positioned over the master vial receiver. This arrangement enables the system to effectively perform the microsphere transfer process.

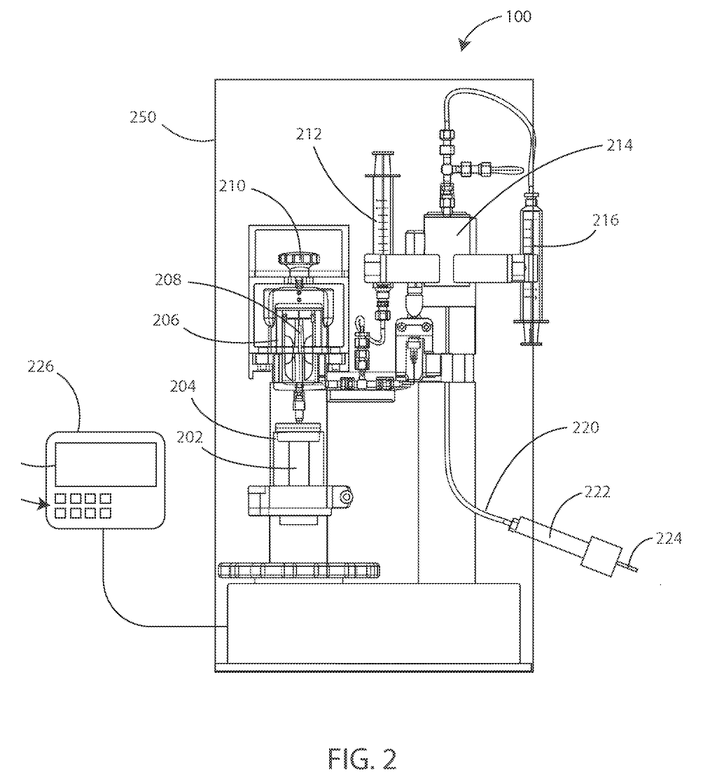

Referring now to FIG. 2, a schematic view of a dose fractionation system in accordance with various embodiments is depicted. The system 100 is configured for the automated fractionation of radioactive microspheres for cancer treatment. The system 100 includes several interconnected components such as a master vial 202, a master vial receiver 204, a mixing container 206, and an agitation system 208. Additionally, the system comprises a carrier fluid pump 212, a holding container 214, a administration pump 216, an output line 220, and a connector hub bypass element 222.

The operation of the system is governed by a control unit 226. The control unit 226 includes a control circuit that is configured to manage and coordinate the functions of the system components. The control circuit may be implemented using a microcontroller, an application-specific integrated circuit (ASIC), a programmable logic device (PLD), or other suitable electronic processing device. The control circuit can be operatively connected to and capable of controlling components such as one or more pumps (including, for example, a carrier fluid pump 212 and a administration pump 216), valves positioned in fluid paths, the agitation system 208 (and specifically a stir motor 210 thereof), and the display screen 228. The control circuit can receive input signals from user input elements 230, such as buttons, switches, or a touchscreen interface, allowing an operator to select, initiate, or adjust system operations. In other embodiments, inputs can be received via wired or wireless communication components. The control circuit can be further configured to send signals to the display screen 228 to provide visual feedback and system status information to the operator. In other embodiments, the control circuit can be configured to send signals via wired or wireless communication components. In addition, the control circuit can process input from various sensors, including radiation sensors or fluid level sensors, and make real-time adjustments to system operation based on detected conditions. Through this architecture, the control unit 226 enables automated and precise control over fluid transfers, agitation cycles, and other functions necessary for preparing and administering fractionated doses of radioactive microspheres, while providing an accessible interface for operator oversight and manual intervention if needed.

The agitation system 208 can incorporate a stir motor 210 to facilitate the mixing process within the mixing container 206, ensuring that the suspension of microspheres and carrier fluid within the mixing container 206 is homogenous.

The connector hub bypass element 222 can include a bypass connection component such as a needle-like structure 224, which can be utilized to minimize dead space in the area of the connection between the system and a delivery catheter which may otherwise collect microspheres thereby enhancing the precision of dose delivery.

The master vial receiver 204 can be designed to securely hold a master vial 202 that contains radioactive microspheres. The mixing container 206 can be placed over the master vial receiver 204. The agitation system 208 can be, at least partially, located within the mixing container 206 (e.g., components thereof such as a paddle, a stir bar, an impeller, or similar structure) and can be capable of rotating and reversing its rotation direction periodically to maintain a high level of homogeneity within the suspension without creating vortex-like conditions within the mixing chamber which may otherwise hinder accurate and precise withdrawal of volumetric portions to the holding container 214. The carrier fluid pump 212, in some cases along with various valves, facilitates the movement of the suspension of microspheres and carrier fluid from the mixing container 206 to the holding container 214.

The output line 220 connects to the holding container 214 and carries the microspheres and carrier fluid to a delivery catheter. As described above, the connector hub bypass element 222 is designed to minimize or eliminate dead space at the point of connection between the fractionation system and the delivery catheter.

In various embodiments, the mixing container 206 is positioned directly above the master vial receiver 204, such as sitting on the master vial receiver 204. While not intending to be bound by theory, positioning the mixing container 206 directly on top of the master vial and the master vial receiver 204 provides several operational and structural advantages that can improve system efficiency and reliability during fractionation of radioactive microsphere doses. The vertical arrangement can minimize the distance and the number of fluidic interfaces between the master vial and the mixing container, thereby reducing the risk of microsphere loss or capture within transfer lines during the carrier fluid flush cycles. Additionally, the direct stacking of the mixing container 206 over the master vial receiver 204 enables a compact system design, which can reduce the system's spatial footprint and simplify physical integration within clinical environments, such as interventional radiology suites. Also, this compact layout aids in maintaining a closed, contained transfer pathway, which can serve to limit the potential for accidental exposure of radioactive microspheres to the external environment.

In various embodiments, radiation shielding 250 can be used to reduce or block the exposure of system operations to radiation from radioactive microspheres in the automated dose fractionation system. For example, radiation shielding 250 can be positioned around one or more of the master vial 202, the master vial receiver 204, the mixing container 206, and/or the holding container 214 to limit occupational exposure to radiation by the operator or bystanders. In some embodiments, radiation shielding 250 can be integrated as a permanent feature of the system housing, while in other embodiments, shielding 250 can be provided as removable or repositionable panels adapted for installation or removal based on procedural requirements or clinical workflow.

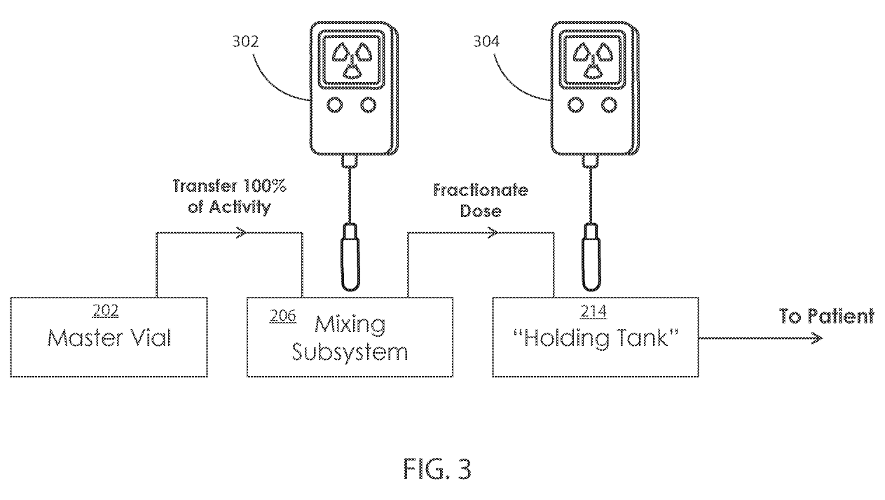

Referring now to FIG. 3, a diagram illustrating various operations within the dose fractionation system for radioactive microspheres is depicted according to various embodiments herein. The automated dose fractionation system 100 is configured for cancer treatment applications. As before, the system 100 includes a master vial 202, which stores the radioactive microspheres. The master vial 202 can be placed into a master vial receiver 204 by a system user. A mixing container 206 is included where the microspheres are combined with a carrier fluid to form a suspension. As indicated in the diagram, the entire dose of radioactive microspheres can be transferred from the master vial 202 to the mixing container via a series of flush cycles.

Each flush cycle can include using a predetermined volume of carrier fluid to mix with the microspheres within the master vial 202 can then convey them along into the mixing container 206. In some embodiments, the amount of fluid with each flush cycle can be about 1, 2, 3, 5, 10, 15, 20, 30, 40, 50, 75 or 100 milliliters (mL) or an amount falling within a range between any of the foregoing. In some embodiments, the flush cycle can be repeated to ensure that all microspheres have been transferred to the mixing container 206. For example, the system can execute 1, 2, 3, 4, 5, or more flush cycles, or an amount falling within a range between any of the foregoing.

Additionally, the system incorporates a holding container 214, which receives the specified dose of microspheres from the mixing container via a fluid channel that may contain features such as valves to influence the movement of the suspension. As indicated in the diagram, fractionation herein occurs by transferring a portion of the radioactive microspheres from the mixing container 206 to the holding container 214. As described above, this can be performed precisely by ensuring that the known quantity of microspheres in the known amount of carrier fluid in the mixing container 206 is homogeneously mixed. In some embodiments, volumetric control can be achieved with high accuracy by using a pump (e.g., the carrier fluid pump 212 in FIG. 2) to move a precise volume of suspension out of the mixing container 206 and into the holding container 214.

Exemplary pumps used with embodiments herein (such as with carrier fluid pump 212, administration pump 216, as well as other possible pumps) can include, but are not limited to, positive displacement pumps capable of delivering tightly controlled volumes of fluid. In particular embodiments, the pump can be a piston pump or a syringe type pump. Positive displacement pumps suitable for use herein may include single or dual syringe pumps, rotary piston pumps, or peristaltic pumps, each configured to provide precise and repeatable movement of fluid volumes. Syringe pumps operate by linear actuation of a plunger within a cylindrical syringe barrel, typically driven by a motor, such as a stepper motor, under electronic control, enabling the transfer of predetermined volumes of suspension with high accuracy, such as with volumetric errors of less than 1% per cycle over the operating range. Rotary piston pumps function by rotating a piston within a precisely machined chamber to displace fixed measurable volumes of fluid per rotation, and can be operated in phases to achieve continuous or pulsed flow. Peristaltic pumps, which deliver fluid by compression and relaxation of a flexible tube with rollers, can also be used, provided the pump is capable of fine control and minimal pulsation to achieve consistent and controlled transfer of known suspension volumes. In certain embodiments, the selected pump is configured to handle fluid volumes in the range of about 0.5 mL to 100 mL per transfer cycle, depending on system requirements, and is capable of supporting automated operation under the control of the system control circuit.

In some embodiments, negative displacement pumps can also be used in place of, or in addition to, positive displacement pumps. By way of example, negative displacement pumps herein can include vacuum-driven pumps, aspirator-based pumps, or diaphragm pumps that operate by generating a region of sub-atmospheric pressure in the downstream portion of the fluid pathway to draw the microsphere suspension from the mixing container to the holding container or through other portions of the system. Such negative displacement pumps may be controlled by the system’s control circuit in a manner analogous to that of positive displacement pumps to enable precise volumetric transfer.

In various embodiments, the integration of radiation sensors within the automated dose fractionation system enables verification or validation of the radioactive dose contained within specific system components, such as the mixing container and the holding container, and/or can validate the specific fractionated dosage being provided. The incorporation of radiation sensors can provide an independent mechanism beyond the volumetric technique described herein to monitor the radioactivity levels associated with the fractionated doses. Specifically, the radiation sensor readings can be correlated to the calculated or intended radioactive content based on volumetric transfer measurements and radioisotope calibration data obtained from the master vial. By comparing the real-time sensor output with the expected radiation dose for each transferred or fractionated portion, system operators and/or control algorithms can identify deviations from anticipated dose levels, which could possibly arise from factors such as incomplete transfer, microsphere retention in system dead space, aggregation, or unintended loss of microspheres during the fractionation process. This capability supports both prospective and retrospective dose validation by providing measurable confirmation that the dose prepared for each infusion event aligns with the prescribed radioactivity.

The use of radiation sensors is also valuable for process monitoring, quality assurance, and regulatory compliance during the preparation and administration of radioactive microsphere therapies. For example, the dose measured by a radiation sensor within the holding container immediately prior to patient delivery can be logged as an objective record of intended administration, supporting institutional documentation protocols or meeting standards for dose traceability. Additionally, feedback from radiation sensors can be employed by the system’s control unit to adjust process parameters or alert users to anomalies, thereby enhancing safety by reducing the likelihood of administering suboptimal or excessive doses.

A variety of radiation sensor technologies can be used with the automated dose fractionation system, depending on sensitivity requirements, spatial constraints, and compatibility with the system’s radioisotopes. In some embodiments, the system can include solid-state radiation detectors, such as scintillation detectors (e.g., sodium iodide, bismuth germanate, lanthanum bromide or cesium iodide activated with thallium CsI(Tl) crystals coupled to photodetectors), semiconductor-based detectors (e.g., silicon or cadmium zinc telluride diodes), or gas-filled detectors (e.g., Geiger-Müller tubes or proportional counters). Scintillation detectors can be useful for nuclear medicine applications because of their high sensitivity to beta and gamma emissions, offering rapid response and good energy resolution for the types of isotopes present in typical microsphere therapies (such as Yttrium-90 or other beta-emitting sources). Semiconductor detectors, including PIN diodes or cadmium zinc telluride (CZT) arrays, can provide compact form factors and are capable of high-resolution measurements suitable for small physical enclosures such as holding containers or mixing chambers. In some embodiments, a combination of sensor types can be used, for example, incorporating both an external scintillation probe for gross activity assessment and an internal semiconductor sensor for finer dose discrimination. Regardless of sensor type, the radiation sensor is operationally connected to the system’s control circuitry to provide measurements of radioactive emissions for real-time or logged dose validation purposes.

As shown in FIG. 3, the system 100 can incorporate multiple radiation sensors to monitor radiation levels. Specifically, the system 100 includes a first radiation sensor 302 and a second radiation sensor 304. In some embodiments, radiation sensor 302 is configured to detect radiation levels in the mixing container 206. This can allow for real-time monitoring of the total radioactivity present within the mixing container to confirm the proper transfer of radioactive microspheres from the master vial. In some embodiments, radiation sensor 304 can be configured to detect radiation within the holding container 214. This can allow for quantitative verification of the radioactivity associated with each fractionated dose transferred to the holding container, thereby enabling confirmation that the prepared dose corresponds to the prescribed or intended activity prior to administration to the patient.

In some embodiments, additional radiation sensors may be positioned at other locations within the automated dose fractionation system to facilitate enhanced process monitoring and verification of system status. For example, a radiation sensor may be disposed adjacent to, or integrated with, the connector hub bypass element 222 to measure localized levels of radioactivity proximate to critical junctions in the fluid path, permitting assessment of residual microspheres or radioactive content in these connector regions. In certain configurations, a radiation sensor can be attached externally to a housing of the system to enable a user to bring the connector hub bypass element or various connection sites (such as catheter connectors or output line terminations) into close proximity with the sensor for a qualitative or quantitative determination of residual radioactivity. Such a measurement can serve as a confirmation that no radioactive microspheres remain within the connection region prior to disconnecting or exchanging system components (for example, during catheter exchanges), thereby promoting procedural safety and reducing the risk of unintended radioactivity retention within system junctions. In these embodiments, the radiation sensor can be operatively coupled to the system’s control unit to provide user alerts, process status confirmation, or integration into automated interlock logic to inhibit or permit further system operations based on the measured radiation level at the designated monitoring point.

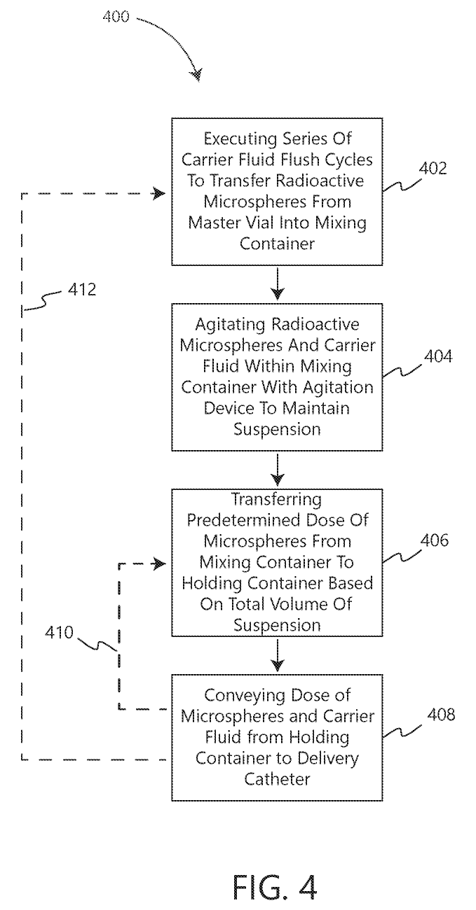

Referring now to FIG. 4, a flowchart is shown that illustrates the sequence of operations in various embodiments. The method for automated dose fractionation 400 of radioactive microspheres for cancer treatment can include various operations. The method can include executing pre-programmed carrier fluid flush cycles 402 to facilitate the transfer of radioactive microspheres from a master vial into a mixing container positioned above the master vial. Next, the method involves agitating the suspension of radioactive microspheres 404 and carrier fluid within the mixing container using an agitation system to ensure homogeneity.

Following agitation, the method entails transferring a predetermined dose (a fractionated dose) of the microsphere suspension from the mixing container to a holding container 406. This is achieved by knowing the amount of microspheres per unit volume of suspension and then controlling the total volume of the suspension being transferred. The method further includes conveying the dose of microspheres and carrier fluid from the holding container to a delivery catheter 408 for administration.

In various embodiments, the transferring and conveying processes are repeated 410 (e.g., multiple fractionated doses) as desired to achieve the treatment objectives for an individual. In various cases, other steps may be taken in between each consecutive transferring and conveying steps. For example, a new delivery catheter may be inserted at a new target site, and then another fractionated dose can be delivered. Alternatively, there may be a pause where a clinician checks various aspects and then another fractionated dose can be delivered to the same site. The transferring and conveying processes can be repeated any desired number of times such as 1, 2, 3, 4, 5, 6, 7, 8 or more times as long as there is activity left, or a number of times falling within a range between any of the foregoing.

In some embodiments, a clinician may want to load another master vial to allow the use of a number of microspheres greater than that contained in one master vial. As such, in some embodiments, the method can include returning 412 to operation 402 to load a new master vial and then proceeding to repeat other operations described herein. By doing so, the total amount of microspheres available for fractionation and subsequent administration exceeds the quantity provided in a single master vial. This approach enables the preparation and administration of larger aggregate doses or the sequential delivery of multiple fractionated doses that may require an amount of microspheres greater than what is supplied in a single master vial. In some embodiments, the system can be configured to prompt the user to load a new master vial, verify correct loading and engagement of the vial and receiver, and then automatically initiate the next series of steps to transfer the contents of the additional master vial into the mixing container.

In some embodiments, the system can be configured to reduce or eliminate dead space along the path of suspension conveyance, particularly in areas such as tubing, connection hubs, valves, or junction interfaces that extend from the mixing container to the output line, holding container, and delivery catheter. Dead space, as used herein, refers to any region in the fluidic pathway where the flow rate is reduced, where recirculation or stagnant fluid can occur, or where the geometry of the pathway creates recesses or voids not efficiently swept by the moving carrier fluid or by microsphere suspension. In these regions, the relatively dense radioactive microspheres may settle under gravity or adhere to surfaces rather than being conveyed with the rest of the suspension, resulting in residual accumulation that is not delivered to the intended treatment site and potentially leading to dosing inaccuracy.

To address this, the fluidic pathways of the system can be designed with uniform internal diameters, short and direct routing, and minimal abrupt transitions or expansions between interconnected components. For example, transfer lines and output tubing may be made as short as practicable and incorporate smooth, radiused connections to avoid spaces where microspheres could be retained. Additionally, connectors such as Luer-locks, Y-connectors, or valved junctions can be selected or engineered with internal geometries that do not create pockets or increases in dead volume. In some embodiments, the connectors can be specifically designed with minimized internal cavities, or are equipped with bypass inserts or flow guides to redirect fluid flow and promote efficient microsphere clearance.

The system may further incorporate specialized components such as a connector hub bypass element at critical connection points, such as at the transition between the system output line and the delivery catheter to reduce dead space. The connector hub bypass element can include features such as a bypass connection needle or line extension that passes through the central lumen of the connector, effectively allowing the suspension to pass through the connection needle or line extension and bypass what would otherwise be an area with dead space within the connector. The bypass element can be configured for manual or automated advancement, such as via a lever, to extend or retract the needle as needed to facilitate both connection and optimized flushing. In some embodiments, the bypass needle projects through the connector hub of the delivery catheter and directly against or into the proximal lumen of the delivery catheter which limits the volume of dead space.

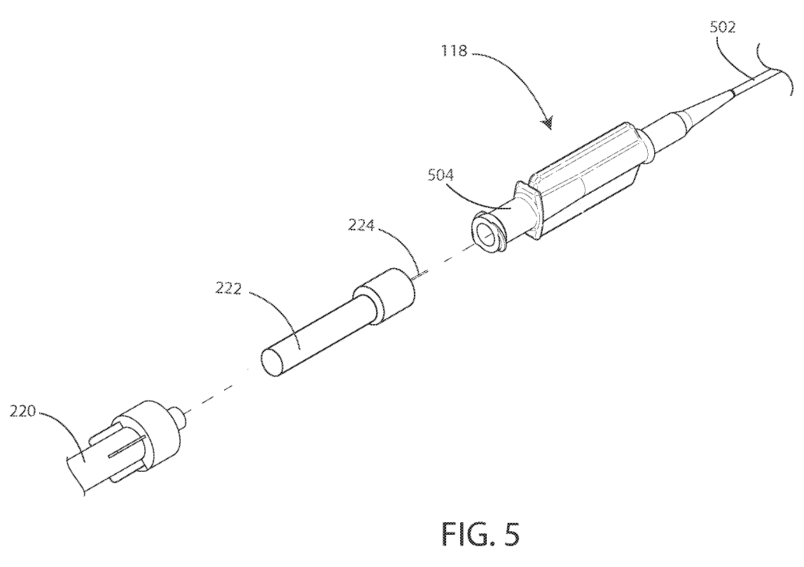

Referring now to FIG. 5, a perspective view illustrates components associated with connecting a fractionation system herein to a delivery catheter 118 according to various embodiments. As previously described, the automated dose fractionation system can include an output line 220 and a connector hub bypass element 222, which incorporates a bypass connection needle 224. Additionally, the delivery catheter 118 can include a delivery catheter connector 504. The delivery catheter 118 can also include a catheter shaft 502.

In some embodiments, the delivery catheter connector 504 can be configured to establish a fluid communication link between the output line 220 and the delivery catheter 118. In some embodiments, the connection between the output line 220 and the delivery catheter 118 can be direct, but in other embodiments, the connector hub bypass element 222 can be disposed there between eliminating dead space.

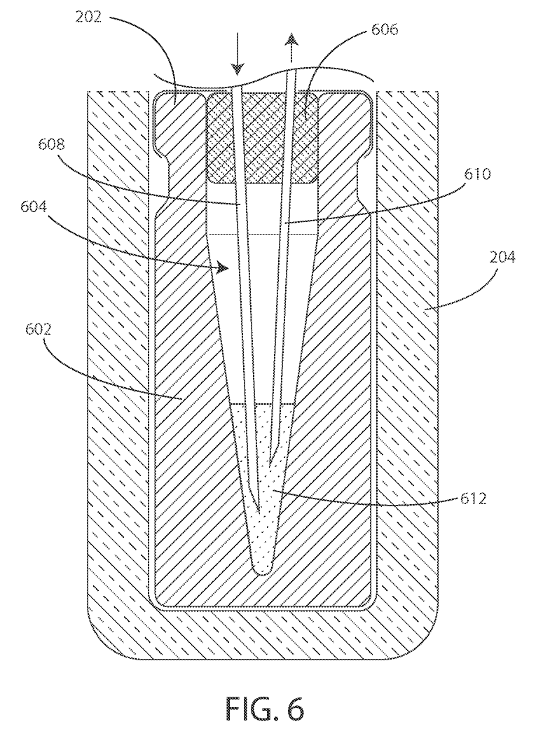

Referring now to FIG. 6, a schematic cross-sectional view of a master vial 202, as held by a master vial receiver 204, in accordance with various embodiments of the invention. The master vial 202 serves as a component for use with an automated dose fractionation system for radioactive microspheres herein. The master vial 202 can include a vial body 602 enclosing an interior volume 604. The master vial 202 can be configured to safely store and facilitate the controlled transfer of radioactive microspheres (such as from the manufacturer) prior to their use in clinical dose preparation. The master vial 202 can be constructed from materials that provide adequate mechanical strength, radiation shielding, and chemical compatibility with both the microspheres and the carrier fluid used in the system. In some embodiments, the vial body 602 can be formed from a glass, such as a borosilicate glass. In some embodiments, the master vial 202 may instead be fabricated from other glass compositions or various polymeric materials.

The top of the interior volume 604 can be secured by a stopper 606. The stopper 606 can include elastomeric materials such as, but not limited to, butyl rubber, chlorobutyl rubber, or fluoropolymer-coated elastomers. These materials can provide an airtight and fluid-tight seal during both long-term storage and as penetrated by fluid injection and withdrawal lines. In some embodiments, the stopper incorporates a self-sealing feature to accommodate multiple punctures by injection and withdrawal needles with minimal risk of loss of sterility or containment. In some embodiments, the stopper can be reinforced with an aluminum or stainless-steel crimp or cap fitted circumferentially over the vial neck to secure the closure.

FIG. 6 also shows a portion of a fluid injection line 608 and a suspension withdrawal line 610. In various embodiments, the fluid injection line 608 is designed to extend into the master vial 202 while the master vial 202 is positioned within the master vial receiver 204. The fluid injection line 608 can be in fluid connection with a carrier fluid pump 212. Likewise, the suspension withdrawal line 610 is configured to extend into the master vial 202 and is in fluid communication with the mixing container 206. The master vial receiver 204 can be configured to securely hold the master vial 202. The master vial receiver 204 can be formed of various materials including, but not limited to, various glasses, polymers, composites, metals, and the like.

In some embodiments, the portions of the fluid injection line 608 and a suspension withdrawal line 610 passing into the master vial 202 can be in the form of a needle, cannula, or metal or glass hypo tube. It will be appreciated, however, that in other areas the fluid injection line 608 and the suspension withdrawal line 610 can transition to flexible or semi-flexible tubing, such as polymeric tubing. In addition, other fluid lines discussed herein can also be formed of flexible or semi-flexible tubing, such as polymeric tubing. FIG. 6 further shows the presence of a radioactive microsphere suspension 612 within the master vial 202.

In various embodiments, the carrier fluid flush cycles include the introduction of a carrier fluid into the master vial 202 via the carrier fluid pump 212. Subsequently, the suspension, comprising the carrier fluid and radioactive microspheres, is withdrawn from the master vial 202 and directed into the mixing container 206. The carrier fluid can be a saline solution, however other biocompatible solutions fit for infusion are also contemplated herein.

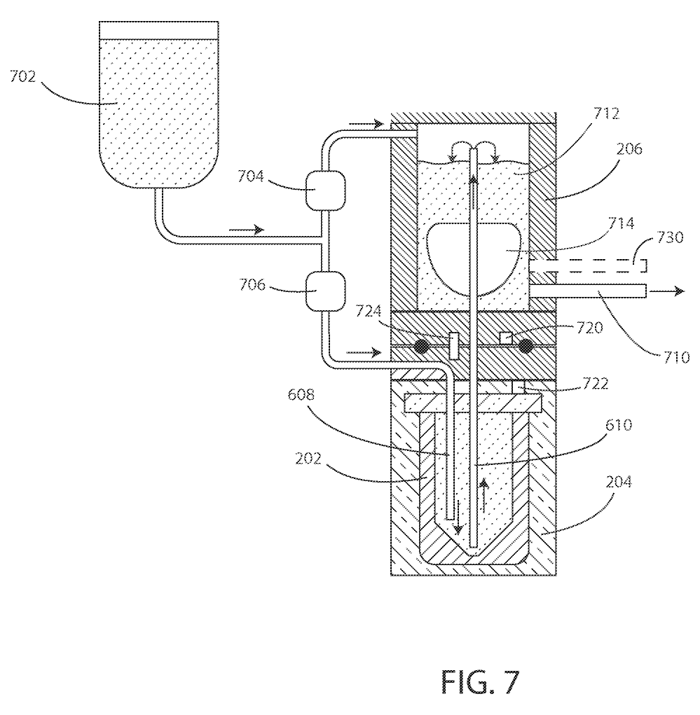

Referring now to FIG. 7, a schematic view is shown of components of the system, including a master vial and a mixing container positioned above the master vial. In FIG. 7, the master vial 202 is held by master vial receiver 204, with the mixing container 206 disposed above the master vial receiver 204. A fluid injection line 608 is configured to deliver saline from saline supply 702 into master vial 202. Suspension withdrawal line 610 provides a pathway for a suspension of radioactive microspheres and carrier fluid to be withdrawn from master vial 202 and transferred into mixing container 206. The system further includes a first pinch valve 704 located on the fluid injection line 608 and a second pinch valve 706 located on the suspension withdrawal line 610. An outflow line 710 is provided for withdrawal of suspension from mixing container 206. Paddle 714 is disposed within mixing container 206 and is configured to agitate and maintain the suspension 712 in a well-mixed state.

To transfer radioactive microspheres from master vial 202 into mixing container 206, first pinch valve 704 is closed and second pinch valve 706 is opened. Saline from saline supply 702 is introduced into master vial 202 through fluid injection line 608. The addition of saline forms a suspension of radioactive microspheres in the master vial 202, and this suspension is drawn through suspension withdrawal line 610 and transferred into mixing container 206. Paddle 714 operates to agitate the suspension 712 within mixing container 206, maintaining the homogeneity of the microspheres in the carrier fluid.

In a subsequent phase, to transfer the well-mixed suspension 712 from mixing container 206 through outflow line 710, first pinch valve 704 is opened and second pinch valve 706 is closed. Saline from saline supply 702 is then delivered into mixing container 206, which displaces the suspension 712 and enables its outflow via outflow line 710. The driving force for transferring the suspension from mixing container 206 through outflow line 710 can be provided by applying positive pressure, negative pressure, or a combination of positive and negative pressures, as required by the specific system configuration.

In some embodiments the system can include various sensors. For example, as shown in FIG. 7, the system can include sensor 720 which can be configured to provide data to the control circuit to detect that the master vial and/or receiver thereof is properly engaged with the mixing container and/or notify a system user of the same. Sensor 720 can be of various types. In some embodiments, sensor 720 can be a mechanical contact sensor, such as a limit switch or microswitch, that is actuated when the master vial receiver is correctly positioned relative to the mixing container. In other embodiments, sensor 720 can be a magnetic sensor, such as a Hall-effect sensor, which detects the presence and alignment of a magnet incorporated in the master vial receiver when proper engagement with the mixing container occurs. In further embodiments, sensor 720 can be an optical sensor, such as an infrared emitter and detector pair, configured to detect closure or alignment of interfacing components. Further, the system can include sensor 722 which can be configured to identify the presence of the master vial and/or the type of vial that has been loaded. Sensor 722 can be of various types. In some embodiments, sensor 722 can be a barcode or matrix code reader configured to read identification labels affixed to the master vial to determine vial type and presence. In other embodiments, sensor 722 can be an RFID (radio-frequency identification) reader configured to detect and interpret a passive RFID tag located on the master vial, thereby confirming vial presence and identifying relevant vial information such as isotope type or activity data.

In various embodiments, the master vial receiver 204 and the mixing container 206 can be mechanically or functionally coupled to ensure secure and stable engagement during system operation. In some embodiments, a drive mechanism can cause movement of the master vial receiver 204 relative to the mixing container 206 to bring them into engagement. The control circuit of the system can be configured control the drive mechanism as well as actuate a locking mechanism, such as a motorized or solenoid-driven latch, clamp, or locking pin assembly, configured to selectively couple the master vial receiver 204 to the mixing container 206 to maintain their positional alignment throughout the procedural workflow. In one embodiment, a locking device 724, under the control of the control circuit, is actuated to transition between an unlocked position, in which the master vial receiver 204 and mixing container 206 may be separated for loading or unloading, and a locked position, in which the master vial receiver 204 is rigidly coupled to the mixing container 206 to prevent unintended separation or misalignment. The engagement or locking status may be verified by a sensor, such as sensor 720, operatively coupled to relay engagement state data to the control circuit for monitoring and feedback, and to provide a notification or interlock to the user interface when engagement is incomplete or if an error condition is detected. This automated locking feature can ensure consistent spatial orientation of the master vial 202 and the mixing container 206, thereby reducing the risk of disconnection, leakage, or accidental exposure during carrier fluid flush cycles or transfer operations. In select embodiments, the control circuit may be further configured to only permit initiation of fluid transfers or agitation cycles once the master vial receiver 204 and mixing container 206 are positively confirmed as locked together. In various embodiments, additional applications of sensor 722 (or other sensors) can be to confirm the presence/absence of interchangeable, interlocking, or configurable components to mitigate setup variation to the validated assumptions of the systems operational algorithms (radiation sensing, volumetric control, etc.).

While FIG. 7 shows the mixing container positioned above the master vial, other orientations are also contemplated herein. By way of example, in some embodiments, the mixing container can be positioned laterally adjacent to the master vial such that the master vial and mixing container reside side-by-side or substantially at the same height relative to the system base. In other embodiments, the mixing container can be positioned below the master vial, permitting gravitational assistance transfer of the suspension from the master vial into the mixing container.

In some clinical scenarios, a system operator can elect to administer a single fractionated dose of radioactive microspheres to a patient, thereby concluding the case without requiring subsequent fractionations or additional administration cycles. In this situation, following completion of the prescribed dose delivery, a residual quantity of radioactive microsphere suspension may remain in the mixing container 206. In consideration of radiation safety and the minimization of unnecessary radiation exposure or material handling, embodiments of the automated dose fractionation system herein can optionally incorporate a dedicated disposal outlet 730 associated with the mixing container 206. The disposal outlet 730 can be configured such that, upon command from the control circuit or operator input via the user interface, the remaining contents of the mixing container 206 can be diverted directly to a shielded waste container (not shown), bypassing the fractionation step and the intermediate holding container 214. This waste management pathway enables the operator to initiate a controlled fluidic transfer, such as by activation of a pump and/or by opening a system valve, to effectively flush the residual radioactive suspension from the mixing container 206 through the disposal outlet 730 and into the shielded waste container. Alternatively, a disposal line can be connected to outflow line 710 or at other points of the system to accomplish the same purpose.

Alternatively, a disposal line can be connected to the disposal outlet of the mixing container so that any residual suspension remaining after fractionated dose delivery can be diverted directly from the mixing container to a shielded waste container. This configuration permits controlled disposal of unused radioactive microspheres and carrier fluid at the end of a treatment procedure. The control circuit can be configured to activate a pump, open a valve, or otherwise enable flow through the disposal line upon receiving a system command or operator input, thereby allowing safe, direct transfer of the suspension from the mixing container into the waste container without passing through the holding container or output line. The disposal line and associated waste management components can be constructed from materials compatible with the radioactive microsphere suspension and provide adequate shielding to limit radiation exposure during handling. In some embodiments, the disposal outlet and disposal line are equipped with connectors or valved fittings that prevent inadvertent leaks or releases of radioactive material during and after the disposal operation. This feature supports the safe handling and containment of radioactive waste in compliance with institutional protocols and regulatory requirements.

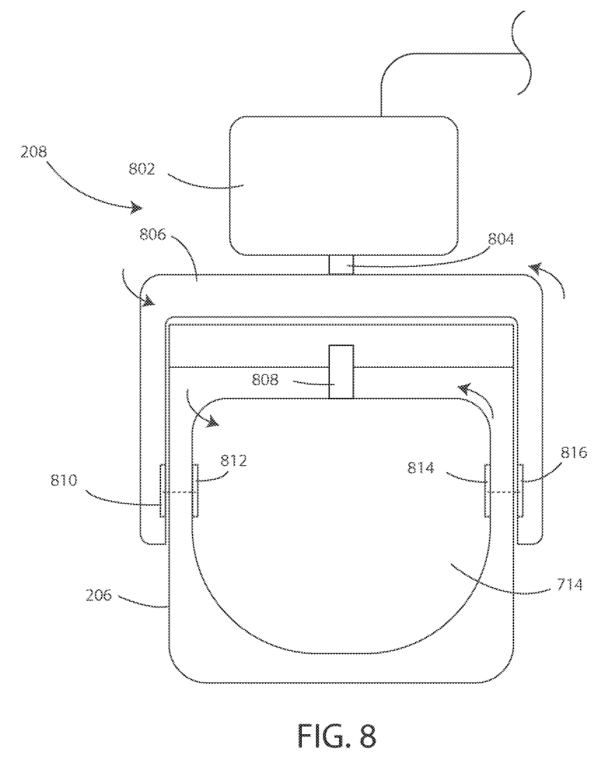

Referring now to FIG. 8, a schematic view of a mixing container 206 and associated agitation system 208 is shown in accordance with various embodiments herein. In the illustrated implementation, the agitation system 208 includes components including a stir motor 802, a rotational drive shaft 804, a magnetic coupling rotor 806, a paddle support shaft 808, and a paddle 706. The motor 802 is operatively coupled to the magnetic coupling rotor 806 via the drive shaft 804 to impart rotational motion. The magnetic coupling rotor 806 includes one or more magnets 810, 816, which are positioned to magnetically couple through a wall of the mixing container 206 with corresponding magnets 812, 814 positioned on the paddle support shaft 808 or paddle 706 disposed within the mixing container 206. Through this magnetic coupling interface, rotation of the yoke 806 by the motor 802 is transferred without the need for physical penetration of the container 206 wall, enabling isolated operation of the interior paddle 706.

This configuration supports complete physical isolation between the interior of the mixing container 206, where suspension of radioactive microspheres is maintained, and the external drive components, such as the motor 802, yoke 806, and drive shaft 804, thereby permitting the mixing container 206 and internal paddle assembly (including support shaft 808 and paddle 706) to be configured as a single-use or disposable set, while allowing reuse of costlier mechanical and electrical elements such as the motor and yoke. The sealed architecture reduces the risk of inadvertent environmental contamination with radioactive material and streamlines system cleaning and maintenance procedures.

Although the illustrated embodiment depicts a magnetically coupled rotational drive, it will be appreciated that alternative actuation techniques for agitating the suspension within the mixing container 206 are contemplated herein. For example, direct mechanical coupling via a sealed rotary joint, oscillatory electromagnetic drive systems, piezoelectric actuators, or non-contact ultrasonically-induced agitation may be employed in other embodiments to provide adequate mixing and homogeneity of the suspension while minimizing contamination risks.

The mixing container 206 can be formed of various materials. By way of example, the mixing container 206 can be constructed from polymeric materials such as polycarbonate, polypropylene, or polyethylene terephthalate (PET), or from glass, such as borosilicate glass. In some embodiments, the mixing container 206 can be fabricated from a composite material that incorporates both polymer and metal components to provide enhanced mechanical durability and chemical resistance. In some embodiments, the mixing container 206 can be transparent to allow visual verification of suspension homogeneity and fluid level during operation, as well as to facilitate inspection for residual microspheres or air bubbles within the container prior to or during fractionation procedures. In other embodiments, the container can be translucent or include a visual inspection window formed as part of the wall structure.

The paddle 706 or other structure can be physically configured to effectively promote and maintain homogeneity of the suspension of radioactive microspheres and carrier fluid within the mixing container 206. The paddle 706 can be configured with one or more blades or vanes, each extending radially from a central hub of the paddle and oriented to sweep through the bulk of the suspension during operation. The cross-sectional shape of the paddle blades can be rectangular, elliptical, or tapered, and can be planar or provided with a curvature to augment fluid movement. For example, in some embodiments, the paddle 706 can include one, two, three, or four blades, and in the case of multiple blades each can be spaced at regular intervals around the central hub and positioned at angles of 90 degrees, 120 degrees, or 180 degrees relative to adjacent blades, respectively, depending on the overall configuration of the mixing container 206. In some variants, the blades are twisted or set at a pitch angle relative to the plane of rotation, forming a helical or propeller-like geometry that can induce axial and/or radial mixing flows. Alternatively, the paddle 706 can include apertures or perforations to facilitate passage of fluid and reduce resistance during rotation, which can assist in minimizing the creation of vortex or cyclonic conditions that could otherwise result in uneven distribution or rapid settling of microspheres away from the withdrawal port of the container.

In various embodiments, the paddle 706 (or similar structure) can be dimensioned so that its blades extend close to, but do not contact, the inner wall of the mixing container 206, thereby increasing agitation within the entire suspension volume while limiting or entirely preventing physical contact with the container. In some embodiments, the length of each paddle blade and the vertical position of the paddle along the drive shaft 704 can be specified to ensure that mixing effectiveness is maximized throughout the vertical extent of the suspension. In some embodiments, multiple paddles or impeller stages may also be positioned on a single drive shaft 704 for enhanced vertical mixing when deeper suspension volumes are processed.

The operation of the agitation system 208 can be governed by the control circuit of the system, which can be configured to modulate parameters such as the rotational speed of the paddle 706, periodic reversal of the paddle 706, wait or pause times between switching directions (e.g., rotate for 5 seconds in direction A, stop for 2 seconds, then rotate for 5 seconds in direction B, then stop for 2 seconds and repeat the cycle), the total duration of the agitation process, the sequence or pattern of rotation cycles executed (including alternating between periods of continuous and intermittent mixing), the acceleration or deceleration rates at the start or end of each rotation interval, and/or the responsiveness of agitation based on real-time sensor feedback such as turbidity, suspension homogeneity, microsphere concentration, or temperature.

In various embodiments, the control circuit can execute pre-set or user-selectable agitation profiles. These profiles can include continuous rotation, intermittent rotation, or pulsed mixing cycles, each defined by combinations of rotation speed, duration of mixing intervals, rest periods, and reversal timing. Agitation cycles can be coordinated with other process events, such as preceding or following carrier fluid flush cycles, or immediately prior to transfer of a fractionated dose to the holding container, to ensure suspension homogeneity at each critical stage of system operation.

In some embodiments, the rotation speed of the paddle 706 can be set at about 5, 10, 20, 30, 40, 50, 60, 70, 80, 90, 100, 110, 130, 150, 175, or 200 revolutions per minute (rpm), or an amount falling within a range between any of the foregoing. The control circuit can be further programmed to periodically reverse the direction of rotation of the paddle 706, with direction changes occurring at predetermined time intervals (such as every 0.1, 0.5, 1, 2, 5, 10, 20, 30, or 60 seconds, or amount of time falling within a range between any of the foregoing) or after a set number of full or partial rotations. This timing may be static or dynamically adjusted by the control circuit based on sensor feedback, such as readings from fluid level or turbidity sensors, or manual input from the operator. The reversal of rotation direction can serve to disrupt the development of rotational flow patterns such as the formation of a vortex that could otherwise support segregation or settling of the comparatively dense radioactive microspheres, thus supporting maintenance of a uniform suspension.

Beyond paddles, it will be appreciated that other components can be used for maintaining the homogeneity of the suspension including, but not limited to, stir bars, impellers, magnetic stir disks, orbital shakers, oscillating rods, or sonication probes, either alone or in combination. The agitation system may incorporate or utilize one or more of these components depending on the configuration of the mixing container, the physical properties of the radioactive microsphere suspension, and the requirements for effective suspension homogeneity. For example, a stir bar can be magnetically driven by a motorized rotating magnetic field generated externally to the mixing container to achieve consistent mixing without breaching the container integrity. Similarly, an impeller or a set of vanes can be mounted to a drive shaft inserted into or magnetically coupled to the mixing chamber to provide directed agitation. In some embodiments, agitation can be achieved through mechanical vibration or by applying ultrasonic energy via a sonication probe to induce turbulence and dispersion of microspheres within the carrier fluid.

### Methods

Many different methods are contemplated herein, including, but not limited to, methods of providing a cancer therapy, methods of automated dose fractionation of radioactive microspheres, and the like. Aspects of system/device operation described elsewhere herein can be performed as operations of one or more methods in accordance with various embodiments herein. In various embodiments, operations described herein and method steps can be implemented as instructions stored on a non-transitory, computer-readable medium that, when executed by one or more processors, cause a system to execute the operations and/or steps.

In an embodiment, a method of automated dose fractionation of radioactive microspheres for cancer treatment is included. The method can include executing a series of pre-programmed carrier fluid flush cycles to transfer radioactive microspheres from a master vial into a mixing container that is disposed over the master vial. The method can further include agitating the radioactive microspheres and carrier fluid within the mixing container with an agitation system to maintain a suspension of radioactive microspheres and carrier fluid. The method can further include transferring a predetermined dose of microspheres from the mixing container to a holding container based on controlling a total volume of a suspension of microspheres and carrier fluid transferred from the mixing container to the holding container.

In an embodiment, the method can further include rotating and periodically changing rotation direction of a component of the agitation system.

In an embodiment, the method can further include conveying the dose of microspheres and carrier fluid from the holding container to a delivery catheter.

In an embodiment, the method can further include transferring a second predetermined dose of microspheres from the mixing container to the holding container based on controlling a total volume of a suspension of microspheres and carrier fluid transferred from the mixing container to the holding container.

In an embodiment, the method can further include conveying the second dose of microspheres and carrier fluid from the holding container to a delivery catheter.

In an embodiment of the method, transferring the second predetermined dose of microspheres from the mixing container to the holding container can be performed without further executing carrier fluid flush cycles to transfer radioactive microspheres from the master vial into the mixing container.

In an embodiment, the method can further include detecting radiation within the holding container. In an embodiment, the method can further include detecting radiation within the mixing container.

### Microspheres

Microspheres herein can include those with a combination of yttria, alumina, and silica. By way of example, in some embodiments, microspheres herein can include Y₂O₃-Al₂O₃-SiO₂ in a 40:20:40 wt. % ratio. It will be appreciated however, that other types of microspheres are also contemplated herein including those with other materials.

In some embodiments, microspheres can be prepared by combining yttrium-89 with alumina and silica, in some cases also using a flame spheroidization method, and using neutron bombardment to convert Y-89 into the beta emitting radioisotope Y-90. In various embodiments, the amount of beta radiation can exceed 400, 600, 800, 1000, 1500, 2000, 2500, 3000, 4000, 5000, 6000, 7000, 8000, 9000, 10000, or even 12000 Becquerel (Bq) per sphere or higher at the time of activity calibration (recognizing that the amount of radiation will drop after that point as the Y-90 radioisotope decays). In some embodiments, the microspheres can be provided in a master vial with activity of 3 Gigabecquerel (GBq) or lower up to 20 GBq or higher (at calibration time or “reference date and time”). However, in some embodiments, the microspheres can be provided in a vial with activity of less than 3, 2.75, 2.5, 2.25, 2, 1.75, 1.5, 1.25, 1.0, 0.75, 0.5, 0.4, 0.3, 0.35, 0.2, 0.15, 0.1, 0.05, or 0.01 GBq, or less at calibration time, or an amount falling within a range between any of the foregoing. It will be appreciated that the use of other isotopes is also contemplated herein.

It will be appreciated that fractionated dosages can vary based on factors including the type of tumor/tissue to be treated, location of the tumor/tissue to be treated, the number of fractionated doses to be provided, factors specific to a particular patient, and the like. In some embodiments the dosage of the therapy can be less than or equal to 5000 Gray (Gy), 4500 Gy, 4000 Gy, 3500 Gy, 3000 Gy, 2500 Gy, 2000 Gy, 1500 Gy, 1000 Gy, 500 Gy, 400 Gy, 300 Gy, 250 Gy, 225 Gy, 200 Gy, 180 Gy, 150 Gy, 120 Gy, 100 Gy, 90 Gy, 80 Gy, 70 Gy, 60 Gy, 50 Gy, 40 Gy, 30 Gy, or 20 Gy, or an amount falling within a range between any of the foregoing.

The size of the microspheres can be extremely small. In some embodiments, the average diameter of the microspheres can be from about 15 micrometers (μm) to about 35 μm. However, in some embodiments the microspheres can be somewhat smaller or larger.

The density of the microspheres can be quite high. In some embodiments, the density of the microspheres can be above 1.4, 1.8, 2, 2.5, or 3 grams/milliliter (g/mL), such as from 3.1 to 3.5 g/mL, or about 3.3 g/mL. By comparison, the density of water at room temperature is about 0.9978 g/mL. As such, the density of microspheres is much higher than an exemplary carrier fluid such as a saline solution which influences how readily such microspheres can settle out of a suspension.

It should be noted that, as used in this specification and the appended claims, the singular forms "a," "an," and "the" include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase "configured" can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An automated dose fractionation system for radioactive microspheres for cancer treatment comprising:
a control circuit;
a carrier fluid pump, wherein the carrier fluid pump is configured to be controlled by the control circuit;
a master vial receiver, wherein the master vial receiver is configured to engage with a master vial containing the radioactive microspheres; and
a mixing container, wherein the mixing container is disposed over the master vial receiver; and
wherein the automated dose fractionation system is configured to execute a series of pre-programmed carrier fluid flush cycles to transfer microspheres from the master vial into the mixing container.

2. The automated dose fractionation system of claim 1, further comprising an agitation system, wherein at least a portion of the agitation system is disposed within the mixing container.

3. The automated dose fractionation system of claim 2, wherein the agitation system is configured to rotate and periodically change rotation direction.

4. The automated dose fractionation system of claim 1, wherein the carrier fluid flush cycles comprise injecting the carrier fluid into the master vial using the carrier fluid pump and then withdrawing a suspension of carrier fluid and radioactive microspheres out of the master vial and into the mixing container.

5. The automated dose fractionation system of claim 1, the radioactive microspheres comprising Y-90 glass microspheres.

6. The automated dose fractionation system of claim 1, wherein the radioactive microspheres have a density greater than three times the density of water.

7. The automated dose fractionation system of claim 1, wherein the mixing container is disposed directly over the master vial receiver.

8. The automated dose fractionation system of claim 1, further comprising a fluid injection line, wherein the fluid injection line is configured to extend into the master vial as held by the master vial receiver and be in fluid communication with the carrier fluid pump.

9. The automated dose fractionation system of claim 1, further comprising a suspension withdrawal line, wherein the suspension withdrawal line is configured to extend into the master vial as held by the master vial receiver and be in fluid communication with the mixing container.

10. The automated dose fractionation system of claim 1, further comprising a holding container; wherein the holding container is in fluid communication with the mixing container; and
wherein the automated dose fractionation system is configured to transfer a predetermined dose of microspheres from the mixing container to the holding container based on controlling a total volume of a suspension of microspheres and carrier fluid transferred from the mixing container to the holding container.

11. The automated dose fractionation system of claim 10, further comprising a radiation sensor, wherein the radiation sensor is configured to detect radiation within the holding container.

12. The automated dose fractionation system of claim 1, further comprising:
a first radiation sensor, wherein the first radiation sensor is configured to detect radiation within the mixing container; and
a second radiation sensor, wherein the second radiation sensor is configured to detect radiation in a holding container.

13. The automated dose fractionation system of claim 1, further comprising an output line, wherein the output line is in fluid communication with a holding container and is configured to convey the microspheres and carrier fluid from the holding container to a delivery catheter that connects to the automated dose fractionation system.

14. The automated dose fractionation system of claim 13, further comprising:
a delivery catheter connector, wherein the delivery catheter connector is configured to provide a fluid communication link between the output line and a delivery catheter; and
a connector hub bypass element, wherein the connector hub bypass element is configured to reduce or eliminate dead space within and between the delivery catheter connector and the delivery catheter.

15. A method of automated dose fractionation of radioactive microspheres for cancer treatment comprising:
executing a series of pre-programmed carrier fluid flush cycles to transfer radioactive microspheres from a master vial into a mixing container that is disposed over the master vial;
agitating the radioactive microspheres and carrier fluid within the mixing container with an agitation system to maintain a suspension of radioactive microspheres and carrier fluid; and
transferring a predetermined dose of microspheres from the mixing container to a holding container in one or more transfer steps based on controlling a total volume of a suspension of microspheres and carrier fluid transferred from the mixing container to the holding container.

16. The method of claim 15, further comprising rotating and periodically changing rotation direction of a component of the agitation system.

17. The method of claim 15, further comprising conveying the dose of microspheres and carrier fluid from the holding container to a delivery catheter.

18. The method of claim 15, further comprising transferring a second predetermined dose of microspheres from the mixing container to the holding container based on controlling a total volume of a suspension of microspheres and carrier fluid transferred from the mixing container to the holding container.

19. The method of claim 18, further comprising conveying the second dose of microspheres and carrier fluid from the holding container to a delivery catheter.

20. The method of claim 15, further comprising detecting radiation within the holding container and/or detecting radiation within the mixing container.
